(19) United States
(12) Patent Application Publication
(10) Pub. No.: US 2021/0407666 A1
(43) Pub. Date: Dec. 30, 2021

(54) SYSTEM FOR TELEMEDICINE DATA TRANSFER

(71) Applicant: Kabushiki Kaisha Topcon, Tokyo (JP)

(72) Inventors: Dawei LI, Oakland, NJ (US); Song MEI, Franklin Park, NJ (US); Zhenguo WANG, Ridgewood, NJ (US); Kinpui CHAN, Ridgewood, NJ (US)

(73) Assignee: Kabushiki Kaisha Topcon, Tokyo (JP)

(21) Appl. No.: 17/357,979

(22) Filed: Jun. 25, 2021

Related U.S. Application Data
(60) Provisional application No. 63/044,512, filed on Jun. 26, 2020.

(51) Int. Cl. G16H 40/67; G06T 7/00; G06T 3/40; G06F 16/53

(52) U.S. Cl. CPC G16H 40/67; G06T 7/0012; G06T 3/40; G06F 16/53; G06T 2207/30168; G06T 2207/20081; G06T 2207/30041; G06T 2207/10101; G06T 2207/20084

(57) ABSTRACT

A telemedicine data transfer system receives telemedicine data from a data source and decides whether to transmit the telemedicine data to a remote end circuit or prevent transmission of the telemedicine data to the remote end circuit based on a result of an evaluation. A telemedicine data transfer system includes image compression and decompression circuits. The decompression circuit may produce decompressed and enhanced telemedicine data. The image compression and decompression circuits include neural networks trained using an objective function to evaluate a difference between a training input data provided to the image compression circuit and a training output data that is output from the image decompression circuit. The training output data is made different from the training input data by a data enhancement operation performed on the training output data prior to the evaluation.

### CROSS-REFERENCE TO RELATED APPLICATIONS

This application claims the benefit of U.S. Provisional Application No. 63/044,512, filed on Jun. 26, 2020, the entire contents of which are incorporated by reference herein.

### FIELD OF THE INVENTION

The present disclosure relates generally to a system that performs transfer of data used in telemedicine.

### BACKGROUND OF INVENTION

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Rising health care costs and general availability of mobile phones and personal computing devices have led to increased interest in telemedicine, including the performance of medical diagnostics and procedures at a physical location separated from medical practitioners and/or traditional medical facilities, such as doctor's offices, out-patient centers, and hospitals.

Telemedical procedures require transfer of data between the patient's location and the location of a medical service provider across private and public data networks including the Internet. Ophthalmic data required for telemedicine may include imagery.

### SUMMARY OF INVENTION

According to one embodiment of the invention, a telemedicine data transfer system includes an image quality assessment circuit to receive telemedicine data from a data source and evaluate the telemedicine data, wherein the image quality assessment circuit is further configured to decide whether to transmit the telemedicine data to a remote end circuit or prevent transmission of the telemedicine data to the remote end circuit based on a result of the evaluation.

In the telemedicine data transfer system, the image quality assessment circuit may be further configured to provide feedback to the data source based on the result of the evaluation and when the image quality assessment circuit decides to prevent transmission of the telemedicine data to the remote end circuit.

In the telemedicine data transfer system, the image quality assessment circuit may include a plurality of deep neural networks, each of the plurality of deep neural networks is trained separately for each different modality, application, and requirement of the telemedicine data.

In the telemedicine data transfer system, the different modalities of the telemedicine data may include at least one of a fundus image, a scanning laser ophthalmoscope, an Optical Coherence Tomography/Optical Coherence Tomography Angiography volume, and a slit lamp digital stream; the different applications of the telemedicine data include at least one of anterior analysis and posterior/retina analysis; and the different requirements of the telemedicine data include at least one of image contrast, image uniformity, motion-free degree of an image, and imaging position.

In the telemedicine data transfer system, when more than one requirement applies to the training of at least one of the plurality of deep neural networks, the training may be performed in two stages including a first stage in which a plurality of sub-deep neural networks, including one sub-deep neural network for each applied requirement, are trained in parallel, and a second stage in which results of each of the plurality of sub-deep neural networks are merged.

In the telemedicine data transfer system, the data source may include one or more of a slit lamp, a fundus camera, and an OCT scanner.

According to another embodiment, a telemedicine data transfer system includes an image compression circuit to receive telemedicine data from a data source and to produce compressed telemedicine data from the received telemedicine data; and an image decompression circuit to receive the compressed telemedicine data via a network and produce decompressed and enhanced telemedicine data from the compressed telemedicine data, the decompressed and enhanced telemedicine data including an enhancement in data quality over the telemedicine data received from the data source. The image compression circuit and decompression circuit include neural networks trained using an objective function to evaluate a difference between a training input data provided to the image compression circuit and a training output data that is output from the image decompression circuit, where the training output data is made different from the training input data by a data enhancement operation performed on the training output data prior to the evaluation by the objective function. The data enhancement operation performed on the training output data is correlated to the enhancement in data quality produced by the image decompression circuit.

In the telemedicine data transfer system, an amount of data enhancement performed on the training output data during training may correspond to the amount of enhancement in data quality produced by the image decompression circuit.

In the telemedicine data transfer system, the image decompression circuit may be further configured to produce the decompressed and enhanced telemedicine data including the enhancement in data quality including one or more of a contrast enhancement, a denoise enhancement, a resolution increase, a channel reduction, a skeletonized output, and a segmented output with different regions having different compression levels.

In the telemedicine data transfer system, the image compression circuit may be further configured to assess an importance of different portions of an image included in the telemedicine data and prioritize sending of a higher importance portion of the image first before sending a lower importance portion of the image.

In the telemedicine data transfer system, the telemedicine data may include at least one of a 2D image and a 3D image.

According to another embodiment, a telemedicine data transfer system includes a remote user circuit that generates a query; and a remote image retrieval circuit to select one or more images from an image database based on the query received from the remote user circuit, wherein each of the remote user circuit and the remote image retrieval circuit include an identical copy of a plurality of categorized query images.

In the telemedicine data transfer system, the query may include at least one of a keyword and a query image.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

### BRIEF DESCRIPTION OF THE DRAWINGS

The scope of the present disclosure is best understood from the following detailed description of exemplary embodiments when read in conjunction with the accompanying drawings, wherein:

FIG. 1 includes a block diagram according to an embodiment of the invention;

FIG. 2 includes a block diagram according to an embodiment of the invention;

FIG. 3 includes a block diagram according to an embodiment of the invention;

FIG. 4A is an example of an optimal right eye fundus image according to an embodiment of the invention;

FIG. 4B is an example of an out-of-focus left eye fundus image according to an embodiment of the invention;

FIG. 4C is an example of a fundus image having regions of high reflection according to an embodiment of the invention;

FIG. 5 includes a table indicating pre-processing and GT for various criteria according to an embodiment of the invention;

FIG. 6 is a block diagram according to an embodiment of the invention;

FIG. 7 is a block diagram according to an embodiment of the invention;

FIG. 8 is a block diagram according to an embodiment of the invention;

FIG. 9 is a block diagram according to an embodiment of the invention;

FIG. 10 shows an example of image retrieval according to an embodiment of the invention;

FIG. 11 is a block diagram of training and retrieving stages according to an embodiment of the invention; and

FIG. 12 is a block diagram of a hardware implementation structure of an embodiment of the invention.

Further areas of applicability of the present disclosure will become apparent from the detailed description provided hereinafter. It should be understood that the detailed description of exemplary embodiments is intended for illustration purposes only and are, therefore, not intended to necessarily limit the scope of the disclosure.

### DETAILED DESCRIPTION

Ophthalmic data for telemedicine typically includes imagery that may occupy a large amount of data. Therefore, to obtain timely operation of telemedical procedures, efficient data transfer over the Internet is important. For example, to perform interoperative checking (i.e., transfer telemedical information during a surgical procedure) it is important to minimize data latency. Thus, high transfer bandwidth and large storage space may be required. Further, image acquisition, transfer, access and storage are each critical stages in the telemedical process, as imagery tends to increase in resolution and frame rate.

Image assessment provides a means for deciding whether an acquired image is useful and should be used (i.e., stored and/or transferred) or whether transfer of the acquired image to a destination should be prevented. Preventing transfer of imagery that is not useful may reduce the data transfer burden on the telemedicine system. Thus, an embodiment of the present invention may include a data assessment framework based on deep learning to assess images and automatically retain only the desired images. The images may be captured for medical purposes by one or more acquisition devices or sensors. If an acquisition device in the telemedicine system needs guidance when acquiring data, the assessment framework may generate a feedback instruction to the acquisition device. For example, the assessment framework may generate one or more (e.g., a sequence of consecutive) instructions to modify a position (or positional relationship to the subject) from which the acquisition device captures the image, such as movement instructions left/right, up/down, back/forward. The guidance may result in automatic repositioning by an acquisition device so equipped, or may result in instructions being provided to an operator for manual repositioning. Alternatively, the generated guidance may indicate another change in an image capturing condition, such as exposure, timing, aperture, pre-processing settings, etc. Further, the assessment framework may be applied to applications such as acquiring color fundus images and Optical Coherence Tomography/Optical Coherence Tomography Angiography (OCT/OCTA) volumes, for example using an OCT scanner.

An embodiment of the invention may include a data compression framework to customize data compression/decompression and thereby reduce bandwidth and data storage space, while also providing an additional layer of data security. For example, compression according to conventional methods may use well known compression protocols (e.g., JPEG) allowing universal compression/decompression. However, an embodiment according to the present invention uses customized compression/decompression modules and algorithms that may be tailored to the data types and images being compressed. The data compression and decompression modules are generated using neural network training modules and the resulting compression scheme may not be readily apparent from the compressed data. Additionally, the compression strategies may change over time. Therefore, third parties are generally unable to extract the compressed data. The data compression framework operates based on deep learning methods and allows for changeable compression strategies (i.e., ratios, compression algorithms, and/or data formats) based on training results tuned to particular image types, modalities, applications, and features. The compression framework may output decompressed images with the same content as the original image (i.e., lossless compression) or the output decompressed images may be different from the original image due to applied enhancements, such as contrast enhancement, color tuning, and structural enhancement, also based on the deep learning methods. As the data compression framework may customize the data compression strategy to the application, the data compression framework may also serve as a non-generic coding-decoding method to increase transfer security, due to the difficulty of decompressing and restoring the original data without access to the compression/decompression algorithm or software.

For telemedicine scenarios that require real time data transfer (i.e., low latency and high speed), such as intraoperative consulting, an embodiment of the invention includes compression strategies that vary based on available transfer bandwidth of the network. Such an embodiment may not only transfer the original images but may also select and transmit an alternative image having an image style derived from the original image, such as an image after denoising, a skeletonized image, a segmented image with an identified region of interest (ROI, i.e., a data set identified for a particular purpose) and identified key features, to further reduce bandwidth requirements.

For telemedicine scenarios requiring image retrieval from query by remote user, an embodiment of the invention includes searching a server end for the queried images with a two-step strategy: (1) classification as a coarse grained retrieval, and (2) a trained deep neural network for fine grained retrieval. Such an embodiment may transfer a compressed version of the original retrieved image(s), with decompression performed automatically at the remote user's end.

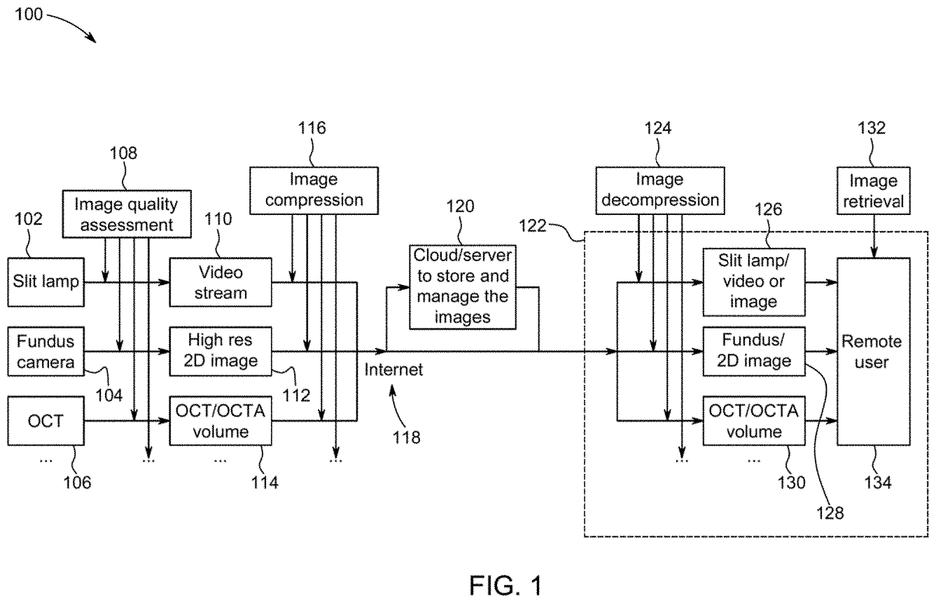

FIG. 1 shows an embodiment of a telemedicine data transfer system 100 according to an embodiment of the invention. Telemedicine data, including images, video and other detected data may be collected from sensors such as, but not limited to, a slit lamp 102, fundus camera 104, or OCT 106. Collected data such as, but not limited to, video stream 110, high res 2D image 112, and OCT/OCTA volume data 114, may be transferred to one or more cloud servers 120 and remote ends 122 via the Internet 118 or other networks. The image quality assessment 108 and image compression 116 may be implemented by one or more processing circuits programmed to perform the corresponding functions. The image decompression 124 and image retrieval 132 may be implemented by one or more processing circuits programmed to perform the corresponding functions.

An image assessment module (image assessment framework) 108 reduces the quantify of the data to be transferred by assessing the collected/acquired image quality, providing guidance for adjustment in acquisition (e.g., in case of bad image quality), and/or select one or more preferred data to transfer (e.g., a best quality image) from amongst a stream or a plurality of collected/acquired data.

An image compression module (image compression framework) 116 reduces the size of data to be transferred, thereby allowing telemedicine procedures to be performed using reduced bandwidth without loss of information required for clinical evaluation.

An image decompression module (image compression framework) 124 decompresses the compressed data with required image quality before further usage to produce, for example slit lamp/video or image(s) 126, fundus/2D image(s) 128, and/or OCT/OCTA volume data/visualizations 130 for a remote user 134. The image decompression module 124 and image compression module 116 are customized together for each image modality (i.e., images of different types, from different imaging sources or imaging application, images having different formats, different subjects, etc.) under different compression strategies (i.e., ratios, compression algorithms, and/or data formats) so that the decompression remains coordinated with the compression.

An image retrieval module 132 retrieves images, video and/or data from the remote end 122 under control of a remote user 134 to provide desired levels of data quality (e.g., with reduced resolution, compression ratio, or field of view) while utilizing reduced bandwidth. One or both of the image decompression module 124 and image retrieval module 132 may be implemented at the remote end 122, or as shown in the example of FIG. 1, may be implemented separately from the remote end 122 (e.g., in a connected computer or server).

The image quality assessment module 108 may perform automatic image assessment. During a telemedicine procedure, multi-modal images may be captured by operators (who may have varying levels of experience) at different sites. Thus, a high percentage of unreadable or otherwise unacceptable images may be produced, for example, for disease diagnosis. In a conventional system, the time to recapture images that satisfy diagnostic needs is time and cost consuming. Thus, the automatic image assessment performed according to the image quality assessment module 108 provides a significant improvement to telemedical procedures.

Conventionally, there are two groups of assessment algorithms: (1) generic image quality processing (e.g., sharpness, contrast, by image histogram, or a combination of factors), or (2) assessment by structural information of the image. However, each of those approaches is limited by imaging condition, morphology and pathology, which results in a variety of disparate and distinguished image assessment modules for different applications under varied imaging modalities and conditions.

The image quality assessment framework according to an embodiment of the invention provides a generic framework within which each of those disparate and distinguished assessment modules can operate to provide a consistent programming and operational interface to other system components. The image quality assessment framework according to the embodiment is achieved through deep learning.

Although a conventional deep learning method based study of assessing image quality has shown high levels of agreement with human graders, for example on rejection/acceptance of acquired fundus images for diabetic retinopathy screening, such a conventional approach takes a long time to assess each image (e.g., 15 sec per image), which may not fulfill the time constraints of telemedicine. According to an embodiment of the invention (1) the criteria for image quality assessment are extended to further include a flexible position, uniformity, high reflection, image contrast, motion etc; and (2) the deep learning model is trained accordingly to achieve such assessment.

According to an embodiment of the invention, the image quality assessment 108 is performed directly on the captured data/images, e.g., immediately after the data is acquired, based on a deep neural network (DNN). Alternatively, the image assessment 108 may be performed on previously stored data.

The image quality assessment framework according to the invention may be adapted separately to different image modalities (e.g., fundus images, scanning laser ophthalmoscope (SLO), OCT/OCTA volume, slit lamp digital stream) on different applications (anterior analysis, posterior/retina analysis). Each application may have its own aspects to assess (requirements), such as image contrast, image uniformity, imaging position and degree to which the image is free from motion artifacts (i.e., motion-free degree). When adapting the framework to assess the images for a specific application, the requirements can be encoded in the pre-processing stage and in the ground truth of the training dataset. The pre-processing and ground truths may be designed for each application separately. Alternatively, portions of images, image capture situations, type of subject, type of disease, type of surgery, etc. . . . ) can be selected permutated or used separately as the criteria of assessment. The selection of criteria purely depends on the requirements. When the assessment has more than one requirement, the model may be trained in two stages: (1) sub-DNNs in parallel work on different requirements, respectively; and (2) a following DNN may merge the results of the sub-DNNs to achieve a final score. The final score may be used (e.g., by comparison to a predetermined or situation dependent threshold) to decide whether to keep a collected image in the assessing stage. The output of each sub-DNN may serve as a feedback signal if necessary to adjust the acquisition devices. In particular, the commands that indicate whether the current live data should be collected (and, if not, how to adjust the machine for collecting/capturing more appropriate data) are encoded in the output of the trained DNNs.

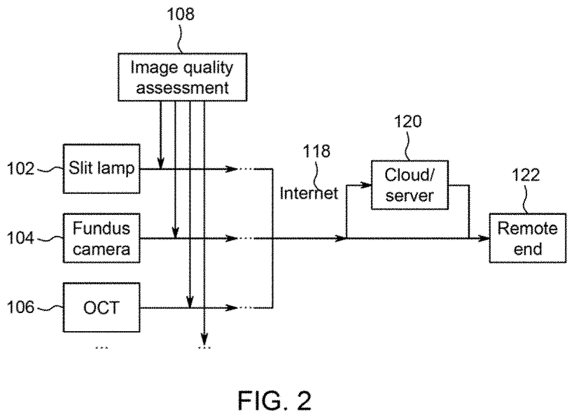

FIG. 2 shows an operation of the image quality assessment 108 in which image assessment is performed on data received from slit lamp 102, fundus camera 104, and OCT 106, and only transferred via the Internet 118 to cloud/server 120 and/or remote end 122 if the image quality assessment 108 determines the image is suitable for use.

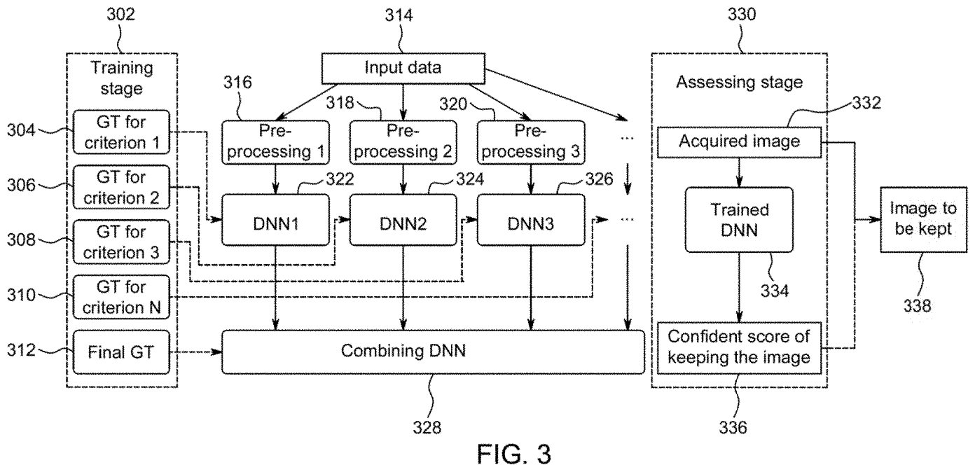

FIG. 3 shows an example of a detailed operation of image quality assessment 108. The network may be customized separately to meet the specific requirements for each application, and each requirement is regulated in a separate deep neural network (DNN1 322, DNN2 324, and DNN3 326) guided by a criterion which can be encoded in ground truth (GT) or preprocessing (PP). In the example of FIG. 3, GT for criterion 1 304 and preprocessing 1 316 guide DNN1 322, GT for criterion 2 306 and preprocessing 2 318 guide DNN2 324, and GT for criterion 3 308 and preprocessing 3 320 guide DNN3 326. For example, when assessing fundus images, criterion 1 may be contrast and uniformity, criterion 2 may be position, and criterion 3 may be reflection level.

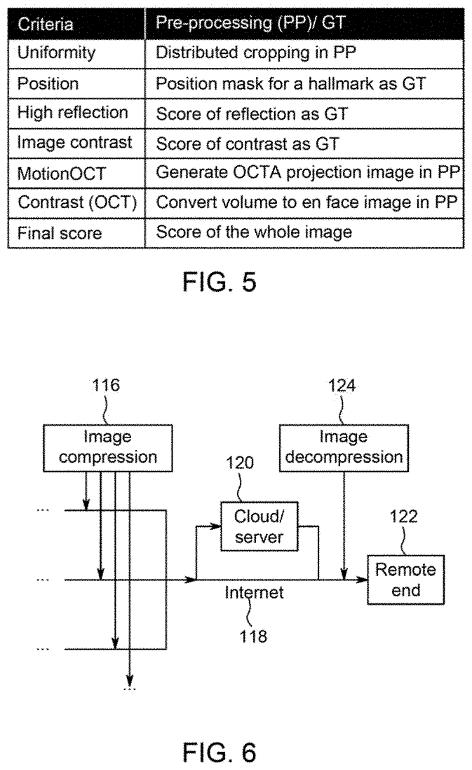

During a training stage 302 an embodiment of the invention may crop each image during the PP stage, into distributed regions 406. The training stage 302 may then use the optimized contrast and uniformity level for each of the regions 408 as the corresponding GT for criteria 1 304. To assess position, an embodiment of the invention labels the optic disc together with the optimized position (right eye differs from left eye) as GT for criteria 2 306. For example, during fundus imaging, the optical nerve head, the retinal vein and/or artery can serve as the target for alignment. To train a DNN, fundus images of optimized relative positions of optical nerve head, the retinal vein and artery in the field of view can serve as positive ground truth. For those with suboptimized relative positions of optical nerve head, the retinal vein and artery in the field of view can serve as negative ground truth, and also with a “suggested” movement (up/down, right/left, back/forward) towards the optimal position. To assess reflection level, an embodiment of the invention may resize the image in a pre-processing (PP) stage and label the image with high/normal as GT for criteria 3 308. In particular, during fundus imaging and corneal imaging, strong back reflection might occur due to the illumination angle and beam shape, which results in strong glare. To train a DNN, images without glare can serve as positive ground truth (to indicate an image to keep). Images with glare can serve as negative ground truth (to indicate an image to abandon). The image size can be reduced by down sampling in prepossessing to achieve a higher processing speed. The description has explained assessment based on position and glare detection as one possible example. However, the invention encompasses using other possible training criteria as well, for example as listed in FIG. 5.

The DNN trained with each criterion comes with an output, respectively. The next step combines the outputs together to get a final output which contains the instructions from each individual output. Thus, the training results of DNN1 322, DNN2 324, and DNN3 326 are combined into a combined and trained DNN 334 by combining DNN 328 based on final GT 312, to make a pass/fail decision for each image.

In the assessing stage 330, the trained DNN 334 is used to assess the data for this application. The input to the trained DNN 334 is acquired images 332 (e.g., fundus images) and the output of the trained DNN 334 is a confidence score of keeping the image 334. The confidence score may be a fraction in the range of 0 (fail) to 1 (pass), which helps to decide whether to keep the image 338.

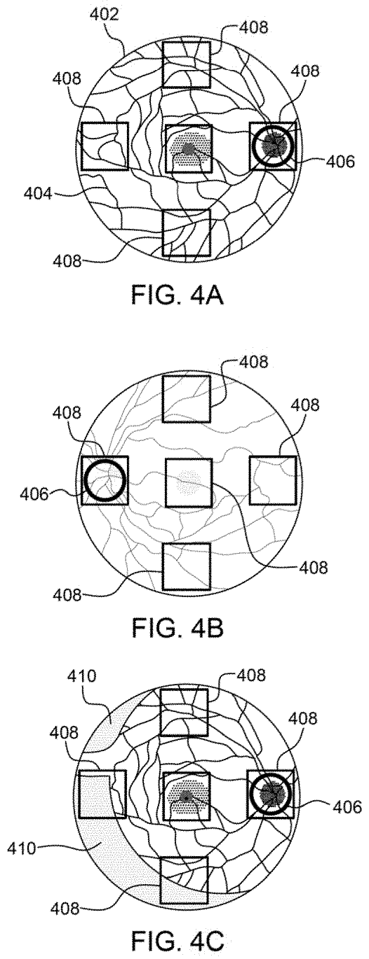

FIG. 4A shows an example of an optimal right eye fundus image. FIG. 4B shows an example of an out-of-focus left eye fundus image. FIG. 4C shows an example of a fundus image having regions of undesirable high reflection 410. Regions 408 for analysis may be automatically or manually selected in advance. In the fundus image examples of FIG. 4A-C, regions 408 are distributed evenly across the entire field of view. Other image modalities may be applied with other strategies for automatically assigning regions 408. To identify a criterion such as uniformity, an embodiment of the invention uses regions 408 to evaluate the intensity of various portions distributed across the entire field of view. But for uniformity in fundus images, for example, it is advantageous to omit evaluation of the region having the optical nerve head, which definitely provides different (e.g., greater or lesser depending on the image modality) reflection than other portions of the fundus image. So, according to an embodiment of the invention, the optical nerve head region, identified by circle 406 may be omitted from the uniformity evaluation. Embodiments of the invention can automatically identify the optical nerve head based on its reflectivity and thereby automatically exclude that region from the evaluation. Embodiments can also automatically identify the regions 408 by spacing them evenly around the image perimeter, with another region in the center. Position markers 406 may also be used to indicate the optical nerve head, the retinal vein and artery, or other features which can serve as targets for alignment.

Thus, according to this example, the system might pass FIG. 4A, while failing FIGS. 4B and 4C. For example, if markers 406 are at appropriate positions for grading by the grader, the lateral position is considered optimal. The markers at the center of FIG. 4B indicate strong back reflection, and if strong back reflection is detected in a fundus, the image is likely to be failed. If the field of view is not uniformly imaged, as in FIG. 4C, the intensity of the five sub field of views will vary beyond a threshold, and the image is likely to fail.

The criteria for training the network may vary for different applications and modalities. For example, in OCT, the system may list OCTA en face as a criterion to assess whether the OCT volume data comes with motion artifacts. If an acquisition device needs feedback from a specific aspect, the output of the subjective function of corresponding DNN (DNN1, DNN2, DNN3, etc. . . . ) can serve as the feedback signal. The DNN trained with each criterion comes with an output, respectively. The next step combines the outputs together to get a final output which contains the instructions from each individual output. The commands that indicate whether the current live data should be collected (and, if not, how to adjust the acquisition device for more appropriate data: e.g., move up/down, left/right, back/forward) are encoded in the output. The framework is flexible to different applications, such as OCT fundus imaging, slit scan fundus imaging, and anterior imaging.

FIG. 5 shows a table indicating pre-processing (PP) and GT for various criteria that can be potentially used in the assessment. The criteria can be used separately and in groups alike. Specifically, to judge whether the image is uniformly imaged, evenly distributed sub fields of view can be selected. The intensity of the sub fields of view may vary beyond a threshold if the whole field of view is not uniformly imaged, in which case the image may be treated as a fail. Position may be the most important aspect to assess during data acquisition. During fundus imaging, optical nerve head, the retinal vein and artery can serve as the target for alignment. To train a DNN, fundus images of optimized relative positions of optical nerve head, the retinal vein and artery in the field of view can serve as positive ground truth. For those with suboptimized relative positions of optical nerve head, the retinal vein and artery in the field of view can serve as negative ground truth, and also with a “suggested” movement (up/down, right/left, back/forward) towards the optimal position. High back reflection usually declines the quality of fundus and corneal images. To train a DNN, images without glare can serve as positive ground truth (to keep), and images with glare can serve as negative ground truth (to abandon). Image contrast is another important factor of image quality. To train a DNN with optimized image contrast, images with contrast above a threshold (depending on specific applications) can serve as positive ground truth (to keep), and images with contrast below the threshold can serve as negative ground truth (to abandon).

FIG. 6 shows an embodiment of the image compression framework in which image compression 116 is performed before transferring data across the Internet 118 or to cloud/server 120, and corresponding image decompression 124 is performed before delivering the data to a remote end 122. Image decompression may be performed by a computer at the remote end 122 or may be performed by a server located on the internet 118. In a telemedicine procedure, separate images/image volumes/videos may be transferred to a remote end 122, which requires high transfer bandwidth. An embodiment of the invention uses customized compression algorithms to reduce the bandwidth required.

Commonly used compression standards, such as BPG, WebP, JPEG and JPEG-2000, cannot achieve the same advantageous compression levels and efficient bandwidth utilization of the embodiments of the invention, because they do are not able to customize compression for the particular image modalities, as in the inventive embodiments. For telemedicine, bandwidth is always a limitation. Traditional compression methods are built for uniformity (i.e., can be applied to every kind of data image format). However, for structuralized clinical data, compression rate according to an embodiment of the invention can be increased based on an understanding of the underlying data structure. There are some regions of interest (e.g., optical nerve head), and other regions are of less/lower importance. Thus, important regions can be compressed less and other regions can be compressed more. Also, since the images are all from the same domain, they may share many common features and feature information, which can be efficiently encoded in a shared dictionary, which allows for greater compression level and less loss of information. Customized methods, such as compressive sensing, provide an alternative solution, however, they involve convex optimization, which is usually computationally expensive to achieve a high reconstruction accuracy. Alternatively, an embodiment of the present invention uses a convolutional neural network for deep learning. In particular, a telemedicine system according to an embodiment of the invention includes a generic image compression framework that applies compression before transferring the data and decompression before using the data. The framework is based on a deep neural network, which can be used to differently and efficiently compress different image modalities (fundus images, SLO, OCT/OCTA volume, slit lamp digital stream . . . ) on various applications (anterior analysis, posterior/retina analysis). Further, the method can compress the images and reduce noise at the same time. For example, if the users wants the reconstructed image of the input to be the original image with reduced noise, an embodiment of the invention can set the target output 710 to be the input data 704 after denoising.

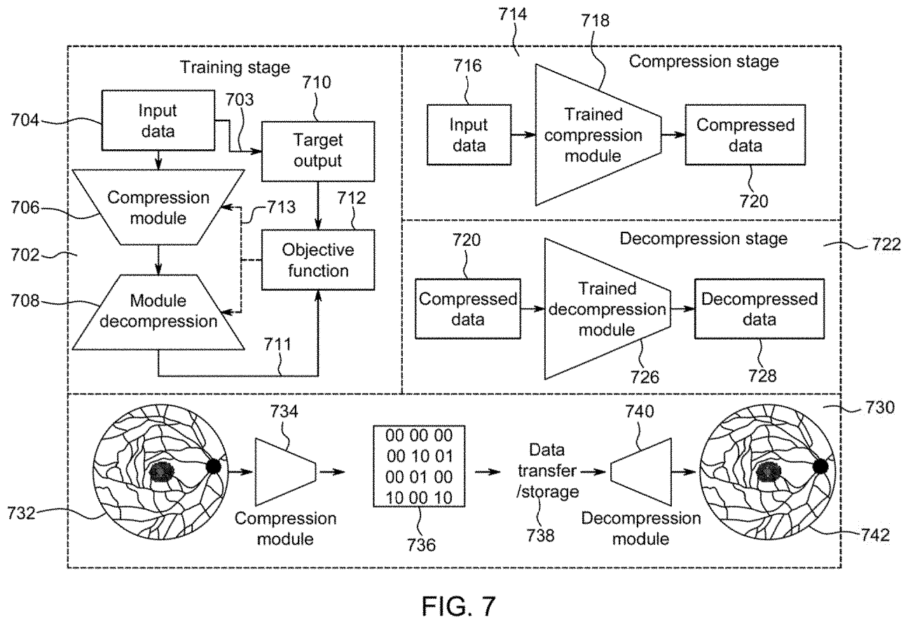

As shown in FIG. 7, in a training stage 702 for each application or modality, a pair of compression/decompression modules 706/708 may be trained together with the feedback generated by an objective function 712. In the training phase, the compression module and decompression module are trained together. During training, the compression module takes original images 704 as input and the decompression module produces reconstructed images 711 as output. The objective function 712 calculates the difference 713 (i.e., error) between a target output image 710 and the and the reconstructed images 711. The difference 713 calculated by the objective function 712 serves as feedback to adjust compression module 706 and decompression module 708 until the error value is stably below a threshold. The objective function 712 may include two parts: a patched decoder to identify the fine-structure difference between the reconstructed images 713 and the original images 704, and an MS-SSIM loss function to evaluate the difference between the input and output on a larger scale, which forces the generator to model the low-frequency information more efficiently.

Thus, the objective function is a training tool to identify differences between original image and reconstructed image. This calculated difference 713 is feedback to the compression/decompression modules 706/708 until the calculated differences is below a threshold. For example, the objective function 712 is a loss function such as a means-square difference function. The parameters of the objective function can be tuned manually for a particular application. “Train a discriminator” is part of the objective function.

According to an embodiment, the target output 710 may intentionally be made different from the input data 704 (which matches the raw data being provided by an acquisition device) by applying an enhancement function 703. For example, the embodiment can increase quality of the target output data 710 by applying denoising and/or other types of image processing as the enhancement function 703, and/or can make the more important regions more clear than less important regions as the enhancement function 703. By tailoring the quality of the target output data 710, and by evaluating the compression/decompression modules using the objective function 712 with respect to the tailored target output 710, the DNN can be trained to take advantage of domain specific advantages to yield better compression/decompression. By improving image quality of the target output data 710 using the enhancement function 703, the trained compression/decompression modules can be trained to automatically perform the same type of image quality enhancement on the decompressed images in addition to the compression. That is, the trained compression and decompression modules may produce an enhancement in the decompressed image that corresponds to (i.e., that correlates with, or is of the same type as) the enhancement performed by the enhancement function 703 during training. Additionally, the degree of enhancement produced in the decompressed image may correspond to the degree of enhancement done by the enhancement function 703 during training. For example, if contrast enhancement is performed by the enhancement function 703, a contrast enhancement effect will also be produced by the trained compression/decompression modules. Thus, the trained system may perform decompression and image enhancement in the same step without requiring a subsequent image improvement processing step.

The objective function 712 takes the target 710 and the output of decompression module 704 as inputs. Input data 704 is the data sent to the DNN for compression target output 710 to indicate the target data format (after decompression). In a conventional training approach, output and input data are often identical (compression usually requires the data before (input) and after (target output) compression to be the same), however, according to an embodiment of the invention, the target output and input data can be made be different based on particular system requirements (i.e., the data after (target output) compression may be different than the input to incorporate desired image processing steps, such as denoising, contrast enhancement, etc.). After the compression and decompression modules are optimized/trained, the compression module is incorporated/delivered to the data acquisition end and the decompression module is incorporated/delivered to the remote end. In a compression stage 714, the trained compression modules 718 are used to compress the original image as input data 716 to generate a binary stream of compressed data 720 which carry the information of the original images. The binary stream of compressed data 720 may be transferred through the network, stored locally, or stored remotely. During a decompression sage 722, the trained decompression models 726, which are delivered to each user's end, may recover decompressed data 728 from the received compressed data 720 before further processing is performed.

In an example 730 of transferring fundus images, an original fundus image 732 is acquired by a fundus camera and the image is compressed by compression module 734 to produce a binary data stream of compressed data 736. The compressed data stream 736 may be stored or transferred 738 to a remote end where a decompression module 740 decompresses the data to produce a decompressed fundus image 742.

The decompressed image may be a loss-less copy of the original image, may be a reduced size version of the original image or may be a further processed version of the original image, for example after performing denoising, contrast enhancement, resolution enhancement, channel reduction, etc on the image.

Information regarding selected compression strategies (e.g., ratios, compression algorithms, and/or data formats) are shared between the compression and decompression modules. A trained decompression model works for all images used in a specific application/modality (e.g., fundus imaging, OCT B-scans, OCT en face images, OCTA images, IR images, RGB images and hyperspectral images). A trained decompression model is sent to the remote end in advance. Subsequently, bit stream 808 including individual images are sent, and the trained decompression model 812 can discern from the raw data stream 808 how to decompress the data stream 808. No additional format info or headers need to be transmitted with the raw data, allowing for further bandwidth reduction. The compression framework works for 2D images, 3D image volumes such as OCT volumes, and/or video streams, as well as other data types.

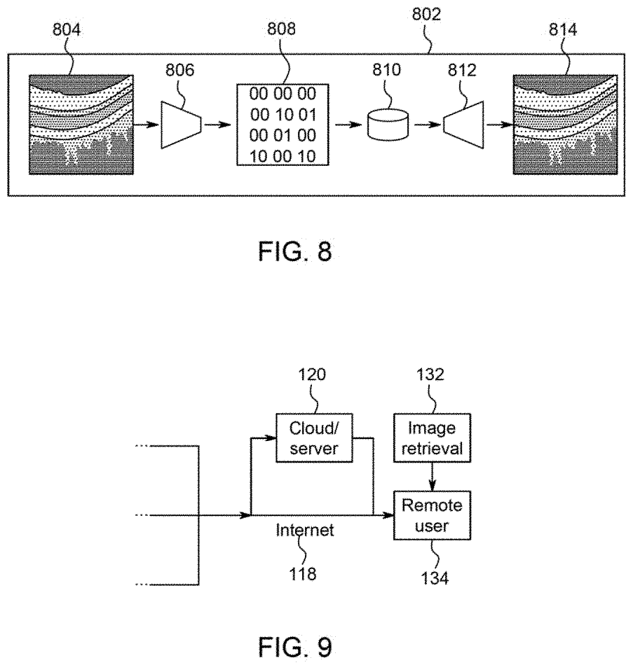

FIG. 8 shows an example of transferring an OCT B-scan image 804 for telemedicine, in which the image is compressed 806 into a binary output stream 808 that may be stored in a memory and/or transferred by a network 810 to a remote end where the data stream may be decompressed/processed 812 to produce a received image 814.

The framework provides the ability to adjust compression strategy based on data transfer bandwidth by training different compression and decompression module pairs with different compression strategies. In this case, the compression-decompression module pairs with different compression ratios are trained separately and delivered to the users in advance. In cases of low bandwidth and high resolution requirement from the user side, break the original image to sub-images and prioritize sending the high importance regions (based on image assessment) first. When transferring data remotely with limited bandwidth, extra time is required. Under this status, the compression/decompression framework provides a solution of transferring a fraction of the whole field of view first. The compression/decompression framework can also be used to store the data locally (for example, serving as the compression method for acquisition devices to save data).

A telemedicine system according to an embodiment of the invention includes an image retrieval system implemented using a computer system for browsing, searching, and retrieving images from a large database of digital images. For example, a medical doctor may remotely refer to various types of imaging modalities individually or simultaneously for the purpose of diagnosis and management of a specific disease in a particular patient.

Conventional remote-image-based diagnosis may be a tedious task due to two reasons: (1) small pathologies in various medical images can be overlooked by doctors due to the limited attention span of the human visual system, and manual image annotation is time-consuming, laborious, and expensive; to address this, there has been a large amount of research done on automatic image annotation. These conventional methods of image retrieval utilize some methods of adding metadata such as captioning, keywords, title, or descriptions to the images so that retrieval can be performed over the annotation words. Alternatively, content-based image retrieval (CBIR) aims at avoiding the use of textual descriptions and instead retrieves images based on similarities in their contents (textures, colors, shapes etc.) to a user-supplied query image or user-specified image features. Most previous attempts show low performance for a massive collection of multimodal databases.

Alternatively, a deep learning based retrieval system for multimodal radiologic images may reach an accuracy and F1.score significantly higher than in conventional medical image retrieval systems.

A telemedicine system according to an embodiment of the invention may include a remote data retrieval framework that takes query images and key words as input/retrieval requests. The retrieval system may operate in two steps: (1) a classification step for coarse grained retrieval, and (2) a trained deep neural networks for fine grained retrieval.

The coarse grained retrieval can be realized by image classification: first categorizing image in terms of image modality and application, then classifying the images by whether it indicates a disease case or not.

For example, when using fundus imaging (image modality) on posterior imaging (application) for diagnosing diabetic retinopathy (DR), image modality and application can be directly recorded when collecting the images. Whether the image is involved in DR can be distinguished by doctors or automatically according to an embodiment of the invention with a trained deep neural network.

Fine grained retrieval further differentiates the images in each category after coarse grained retrieval. Fine grained retrieval is dedicated to deal with more complex situations, such as staging the progression of a complex symptom, which is difficult to describe clearly in simple words, while an image in similar situation (query image) can deliver the messages more explicitly and with greater clarity. A query image serves as an index to search the whole database. The training is performed in advance.

According to an embodiment of the invention, a deep retrieval network includes a tele-ophthalmic image retrieval (TOIR) that is designed to match the images in the database to the query image. The TOIR retrieves images in two steps: first coarse grained retrieval based on classification and then fine grained retrieval based on the deep neural network for retrieval. According to an embodiment of the invention, the deep retrieval network is first trained and then the trained network is used to retrieve raw images. The deep retrieval network can also be trained and then be used to retrieve images with generic compression format and customized compression format. For example, the network can be trained with and retrieve data not only with its original format (such as raw image) but also with compressed data.

When using the TOIR system according to an embodiment of the invention, a remote user sends a request to the server/cloud end; the server/cloud end retrieves the required images and then transfers the compressed matrix of the retrieved images back to the user. Server/cloud end and user end share the same catalogs of classification. Server/cloud end and user end share the compression and decompression pairs for compression and decompressions accordingly.

FIG. 9 shows an example of operation of automatic retrieval 132 according to an embodiment of the invention. Image retrieval is initiated by a remote user 134 providing, to a cloud/server 120 a request to retrieve images of interest. The request may include key words or a query image (as an example to retrieve similar images). In telemedicine system, the database, where the data is stored, usually locates at the server/cloud end—which is separated from the users. Images meeting the request are retrieved at the server end 120 and the retrieved images are delivered to the remote user 134 through the Internet 118.

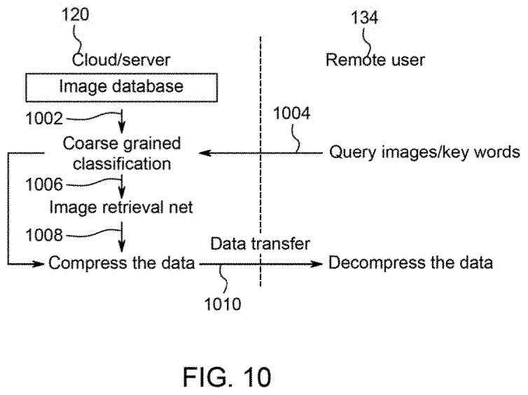

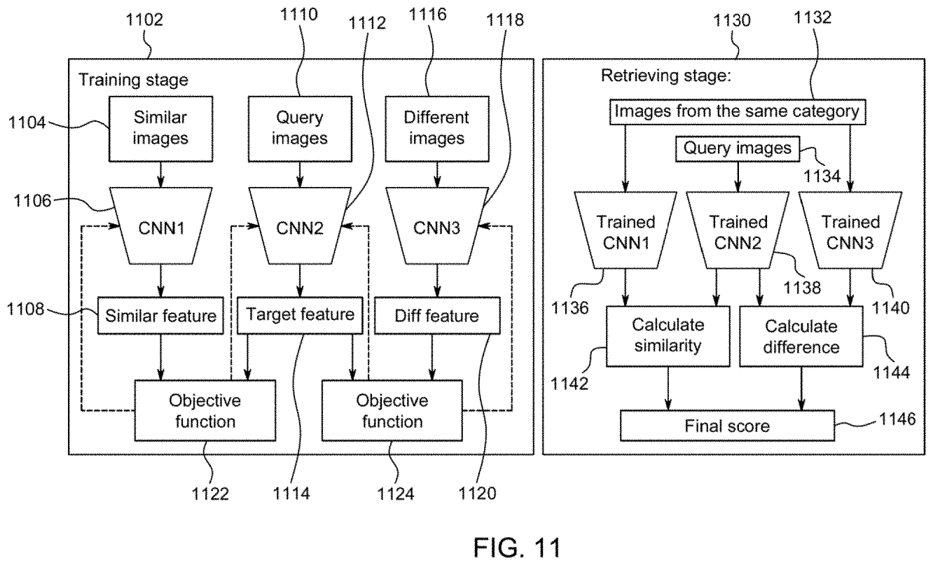

FIG. 10 shows an example of the image retrieval operation according to an embodiment of the invention. To achieve facilitate image retrieval, an embodiment of the invention encodes all available images at the server end 120 into different categories 1002 for coarse grained retrieval. Each category is then indexed with key words and query images 1006. The key words and query images serve as a “dictionary,” which is delivered to and stored at both remote users' end 134 and servers' end 120 in advance. When the “dictionary” is updated, the changes are delivered to the remote users' end 134 accordingly. The remote users 134 start a request by submitting key words (which should be in the dictionary) or query images (which are not necessary in the dictionary) 1004. Thus, in an embodiment of the invention, (1) the two-step retrieval framework comes with DNN for fine grained retrieval; and (2) the DNN designed for retrieval utilizes a combination of calculating both similar and different features. Further, the query image can be compressed in a custom compression data format as discussed above.

If the submitted items (i.e., request commands) are listed in the dictionary, the server will fetch the data that from same category to the submitted items in the database. If the submitted items (query images) are not listed in the dictionary, the server will classify the image into an already established category first (coarse grained retrieval) and then further retrieve the images within the same category in the database with a trained image retrieval net. Transferring the retrieved images requires high transfer bandwidth instantaneously. To reduce the requirement of internet bandwidth, an embodiment of the invention may further compress the raw image 1008 first and then send the compressed data 1010 to the remote users where it can be decompressed before further use.

FIG. 11 shows an example of training stage 1102 and a retrieval stage 1130 included in the image retrieval 132 according to an embodiment of the invention. Images for training and retrieval can be either raw image or images after compression. The network in this example consists of three compression neural network (CNN) modules: CNN1 1106 to extract the features of the images from the similar images dataset 1104, which are similar to query images (where the similarity is decided by the objective function in training stage, and the objective function is selected on a case by case basis), CNN2 1112 to extract the features from query images 1110 provided by user query, and CNN3 1116 to extract the features from images in a different images dataset 1116, which are images that are different from query images (as determined by the objective function in the training stage). The output 1108 of CNN1 1106 and output 1114 of CNN2 1112 are compared by an objective function 1122. The output of the objective function 1122 is used to minimize the difference between the outputs 1108 and 1114 of CNN1 1106 and CNN2 1112, respectively. The outputs 1112 of CNN2 and output 1120 of CNN3 1118 are compared by another objective function 1124. The output of objective function 1124 is used to maximize the difference between the outputs 1114 and 1120 of CNN2 1112 and CNN3 1118, respectively.

After training, CNN1 1106, CNN2 1112, and CNN3 1118 are used as trained CNNs: Trained CNN1 1136, Trained CNN2 1138, and Trained CNN3 1140, respectively. Training is stopped when the error, i.e., the difference between the desired output and the expected output is below a threshold value or the number of iterations or epochs is above some threshold value. The trained CNNs are used in the fine grained retrieval stage. During a retrieval stage 1130, the query image is the input of CNN2; the image from the same category of the query image in the database is the input of CNN1 and CNN3. During image retrieval, the images are first categorized into established categories (coarse grained retrieval) and then further retrieved within the same category with the trained retrieval net (fine grained retrieval). At this stage, the images will serve as the input of trained CNN1 and CNN3, to compare with a group of sample/standard images (query images). CNNs extracts the features of query images and the images from database respectively, and then calculate their similarity and difference. Calculations 1142 and 1144, as well as final score 1146 are each performed by objective functions selected in the training stage. Their similarity and difference are weighted combined to decide whether to send the image to the remote user.

As used herein, an element or step recited in the singular and proceeded with the word “a” or “an” should be understood as not excluding plural elements or steps, unless such exclusion is explicitly recited. Furthermore, references to “one embodiment” of the present invention are not intended to be interpreted as excluding the existence of additional embodiments that also incorporate the recited features.

Control methods and systems described herein may be implemented using computer programming or engineering techniques including computer software, firmware, hardware or any combination or subset thereof, wherein the technical effects may include at least processing of the three-dimensional volumetric data and diagnostic metrics according to the present disclosure.

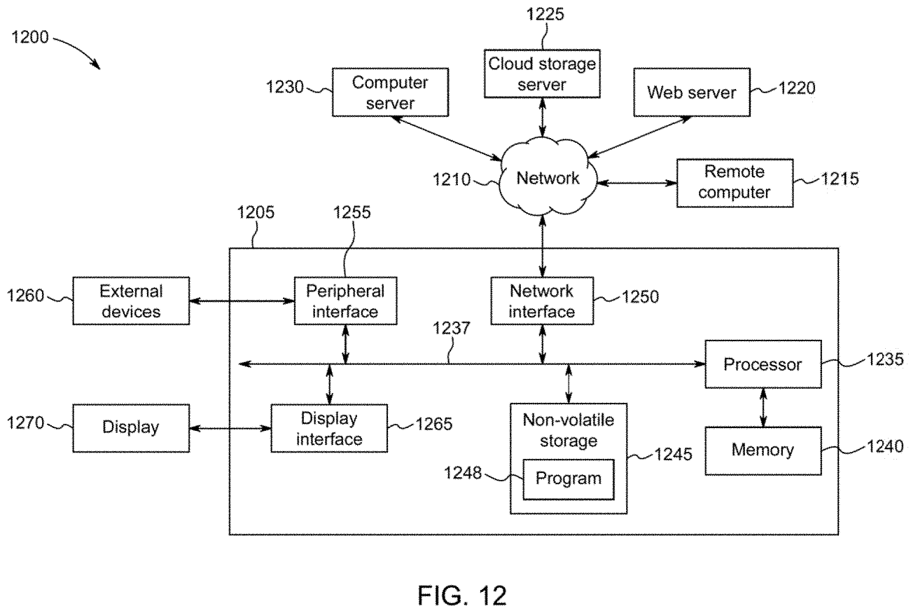

FIG. 12 illustrates a block diagram of a computer that may implement the various embodiments described herein. Control aspects of the present disclosure may be embodied as a system, a method, and/or a computer program product. The computer program product may include a computer readable storage medium on which computer readable program instructions are recorded that may cause one or more processors to carry out aspects of the embodiment.

The computer readable storage medium may be a tangible and non-transitory device that can store instructions for use by an instruction execution device (processor). The computer readable storage medium may be, for example, but is not limited to, an electronic storage device, a magnetic storage device, an optical storage device, an electromagnetic storage device, a semiconductor storage device, or any appropriate combination of these devices. A non-exhaustive list of more specific examples of the computer readable storage medium includes each of the following (and appropriate combinations): flexible disk, hard disk, solid-state drive (SSD), random access memory (RANI), read-only memory (ROM), erasable programmable read-only memory (EPROM or Flash), static random access memory (SRAM), compact disc (CD or CD-ROM), digital versatile disk (DVD), MO, and memory card or stick. A computer readable storage medium, as used in this disclosure, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions implementing the functions described in this disclosure can be downloaded to an appropriate computing or processing device from a computer readable storage medium or to an external computer or external storage device via a global network (i.e., the Internet), a local area network, a wide area network and/or a wireless network. The network may include copper transmission wires, optical communication fibers, wireless transmission, routers, firewalls, switches, gateway computers and/or edge servers. A network adapter card or network interface in each computing or processing device may receive computer readable program instructions from the network and forward the computer readable program instructions for storage in a computer readable storage medium within the computing or processing device.

Computer readable program instructions for carrying out operations of the present disclosure may include machine language instructions and/or microcode, which may be compiled or interpreted from source code written in any combination of one or more programming languages, including assembly language, Basic, Fortran, Java, Python, R, C, C++, C#, or similar programming languages. The computer readable program instructions may execute entirely on a user's personal computer, notebook computer, tablet, or smartphone, entirely on a remote computer or computer server, or any combination of these computing devices. The remote computer or computer server may be connected to the user's device or devices through a computer network, including a local area network or a wide area network, or a global network (i.e., the Internet). In some embodiments, electronic circuitry including, for example, programmable logic circuitry, field-programmable gate arrays (FPGA), or programmable logic arrays (PLA) may execute the computer readable program instructions by using information from the computer readable program instructions to configure or customize the electronic circuitry, in order to perform aspects of the present disclosure.

Aspects of the present disclosure are described herein with reference to flow diagrams and block diagrams of methods, apparatus (systems), and computer program products according to embodiments of the disclosure. It will be understood by those skilled in the art that each block of the flow diagrams and block diagrams, and combinations of blocks in the flow diagrams and block diagrams, can be implemented by computer readable program instructions.

The computer readable program instructions that may implement the systems and methods described in this disclosure may be provided to one or more processors (and/or one or more cores within a processor) of a general purpose computer, special purpose computer, or other programmable apparatus to produce a machine, such that the instructions, which execute via the processor of the computer or other programmable apparatus, create a system for implementing the functions specified in the flow diagrams and block diagrams in the present disclosure. These computer readable program instructions may also be stored in a computer readable storage medium that can direct a computer, a programmable apparatus, and/or other devices to function in a particular manner, such that the computer readable storage medium having stored instructions is an article of manufacture including instructions which implement aspects of the functions specified in the flow diagrams and block diagrams in the present disclosure.

The computer readable program instructions may also be loaded onto a computer, other programmable apparatus, or other device to cause a series of operational steps to be performed on the computer, other programmable apparatus or other device to produce a computer implemented process, such that the instructions which execute on the computer, other programmable apparatus, or other device implement the functions specified in the flow diagrams and block diagrams in the present disclosure.

FIG. 12 is a functional block diagram illustrating a networked system 1200 of one or more networked computers and servers. In an embodiment, the hardware and software environment illustrated in FIG. 12 may provide an exemplary platform for implementation of the software and/or methods according to the present disclosure. Referring to FIG. 12, a networked system 1200 may include, but is not limited to, computer 1205, network 1210, remote computer 1215, web server 1220, cloud storage server 1225 and computer server 1230. In some embodiments, multiple instances of one or more of the functional blocks illustrated in FIG. 12 may be employed.

Additional detail of a computer 1205 is also shown in FIG. 12. The functional blocks illustrated within computer 1205 are provided only to establish exemplary functionality and are not intended to be exhaustive. And while details are not provided for remote computer 1215, web server 1220, cloud storage server 1225 and computer server 1230, these other computers and devices may include similar functionality to that shown for computer 1205. Computer 1205 may be a personal computer (PC), a desktop computer, laptop computer, tablet computer, netbook computer, a personal digital assistant (PDA), a smart phone, or any other programmable electronic device capable of communicating with other devices on network 1210.

Computer 1205 may include processor 1235, bus 1237, memory 1240, non-volatile storage 1245, network interface 1250, peripheral interface 1255 and display interface 1265. Each of these functions may be implemented, in some embodiments, as individual electronic subsystems (integrated circuit chip or combination of chips and associated devices), or, in other embodiments, some combination of functions may be implemented on a single chip (sometimes called a system on chip or SoC).

Processor 1235 may be one or more single or multi-chip microprocessors, such as those designed and/or manufactured by Intel Corporation, Advanced Micro Devices, Inc. (AMD), Arm Holdings (Arm), Apple Computer, etc. Examples of microprocessors include Celeron, Pentium, Core i3, Core i5 and Core i7 from Intel Corporation; Opteron, Phenom, Athlon, Turion and Ryzen from AMD; and Cortex-A, Cortex-R and Cortex-M from Arm. Bus 1237 may be a proprietary or industry standard high-speed parallel or serial peripheral interconnect bus, such as ISA, PCI, PCI Express (PCI-e), AGP, and the like.

Memory 1240 and non-volatile storage 1245 may be computer-readable storage media. Memory 1240 may include any suitable volatile storage devices such as Dynamic Random Access Memory (DRAM) and Static Random Access Memory (SRAM). Non-volatile storage 1245 may include one or more of the following: flexible disk, hard disk, solid-state drive (SSD), read-only memory (ROM), erasable programmable read-only memory (EPROM or Flash), compact disc (CD or CD-ROM), digital versatile disk (DVD) and memory card or stick.

Program 1248 may be a collection of machine readable instructions and/or data that is stored in non-volatile storage 1245 and is used to create, manage and control certain software functions that are discussed in detail elsewhere in the present disclosure and illustrated in the drawings. In some embodiments, memory 1240 may be considerably faster than non-volatile storage 1245. In such embodiments, program 1248 may be transferred from non-volatile storage 1245 to memory 1240 prior to execution by processor 1235.

Computer 1205 may be capable of communicating and interacting with other computers via network 1210 through network interface 1250. Network 1210 may be, for example, a local area network (LAN), a wide area network (WAN) such as the Internet, or a combination of the two, and may include wired, wireless, or fiber optic connections. In general, network 1210 can be any combination of connections and protocols that support communications between two or more computers and related devices.

Peripheral interface 1255 may allow for input and output of data with other devices that may be connected locally with computer 1205. For example, peripheral interface 1255 may provide a connection to external devices 1260. External devices 1260 may include devices such as a keyboard, a mouse, a keypad, a touch screen, and/or other suitable input devices. External devices 1260 may also include portable computer-readable storage media such as, for example, thumb drives, portable optical or magnetic disks, and memory cards. Software and data used to practice embodiments of the present disclosure, for example, program 1248, may be stored on such portable computer-readable storage media. In such embodiments, software may be loaded onto non-volatile storage 1245 or, alternatively, directly into memory 1240 via peripheral interface 1255. Peripheral interface 1255 may use an industry standard connection, such as RS-232 or Universal Serial Bus (USB), to connect with external devices 1260.

Display interface 1265 may connect computer 1205 to display 1270. Display 1270 may be used, in some embodiments, to present a command line or graphical user interface to a user of computer 1205. Display interface 1265 may connect to display 1270 using one or more proprietary or industry standard connections, such as VGA, DVI, DisplayPort and HDMI.

As described above, network interface 1250, provides for communications with other computing and storage systems or devices external to computer 1205. Software programs and data discussed herein may be downloaded from, for example, remote computer 1215, web server 1220, cloud storage server 1225 and computer server 1230 to non-volatile storage 1245 through network interface 1250 and network 1210. Furthermore, the systems and methods described in this disclosure may be executed by one or more computers connected to computer 1205 through network interface 1250 and network 1210. For example, in some embodiments the systems and methods described in this disclosure may be executed by remote computer 1215, computer server 1230, or a combination of the interconnected computers on network 1210.

Data, datasets and/or databases employed in embodiments of the systems and methods described in this disclosure may be stored and or downloaded from remote computer 1215, web server 1220, cloud storage server 1225 and computer server 1230.

Numerous modifications and variations of the present invention are possible in light of the above teachings. It is therefore to be understood that within the scope of the appended claims, the invention may be practiced otherwise than as specifically described herein.

What is claimed is:

1. A telemedicine data transfer system comprising:
an image quality assessment circuit to receive telemedicine data from a data source and evaluate the telemedicine data, wherein
the image quality assessment circuit is further configured to decide whether to transmit the telemedicine data to a remote end circuit or prevent transmission of the telemedicine data to the remote end circuit based on a result of the evaluation.

2. The telemedicine data transfer system according to claim 1, wherein the image quality assessment circuit is further configured to provide feedback to the data source based on the result of the evaluation and when the image quality assessment circuit decides to prevent transmission of the telemedicine data to the remote end circuit.

3. The telemedicine data transfer system according to claim 1, wherein
the image quality assessment circuit includes a plurality of deep neural networks, each of the plurality of deep neural networks is trained separately for each different modality, application, and requirement of the telemedicine data.

4. The telemedicine data transfer system according to claim 3, wherein
the different modalities of the telemedicine data include at least one of a fundus image, a scanning laser ophthalmoscope, an optical coherence tomography/optical coherence tomography angiography volume, and a slit lamp digital stream;
the different applications of the telemedicine data include at least one of anterior analysis and posterior/retina analysis; and
the different requirements of the telemedicine data include at least one of image contrast, image uniformity, motion-free degree of an image, and imaging position.

5. The telemedicine data transfer system according to claim 4, wherein
when more than one requirement applies to the training of at least one of the plurality of deep neural networks, the training is performed in two stages including
a first stage in which a plurality of sub-deep neural networks, including one sub-deep neural network for each applied requirement, are trained in parallel, and
a second stage in which results of each of the plurality of sub-deep neural networks are merged.

6. The telemedicine data transfer system according to claim 1, wherein the data source includes one or more of a slit lamp, a fundus camera, and an optical coherence tomography scanner.

7. A telemedicine data transfer system comprising:
an image compression circuit to receive telemedicine data from a data source and to produce compressed telemedicine data from the received telemedicine data; and
an image decompression circuit to receive the compressed telemedicine data via a network and produce decompressed and enhanced telemedicine data from the compressed telemedicine data, the decompressed and enhanced telemedicine data including an enhancement in data quality over the telemedicine data received from the data source, and
the image compression circuit and decompression circuit including neural networks trained using an objective function to evaluate a difference between a training input data provided to the image compression circuit and a training output data that is output from the image decompression circuit, where the training output data is made different from the training input data by a data enhancement operation performed on the training output data prior to the evaluation by the objective function, the data enhancement operation performed on the training output data being correlated to the enhancement in data quality produced by the image decompression circuit.

8. The telemedicine data transfer system according to claim 7, wherein an amount of data enhancement performed on the training output data during training correspond to the amount of enhancement in data quality produced by the image decompression circuit.

9. The telemedicine data transfer system according to claim 7, wherein the image decompression circuit is further configured to produce the decompressed and enhanced telemedicine data including the enhancement in data quality including one or more of a contrast enhancement, a denoise enhancement, a resolution increase, a channel reduction, a skeletonized output, and a segmented output with different regions having different compression levels.

10. The telemedicine data transfer system according to claim 7, wherein the image compression circuit is further configured to assess an importance of different portions of an image included in the telemedicine data and prioritize sending of a higher importance portion of the image first before sending a lower importance portion of the image.

11. The telemedicine data transfer system according to claim 7, wherein the telemedicine data includes at least one of a 2D image and a 3D image.

12. A telemedicine data transfer system comprising:
a remote user circuit that generates a query; and
a remote image retrieval circuit to select one or more images from an image database based on the query received from the remote user circuit,
wherein each of the remote user circuit and the remote image retrieval circuit include an identical copy of a plurality of categorized query images.

13. The telemedicine data transfer system according to claim 13, wherein the query includes at least one of a keyword and a query image.
